(19) United States
(12) Patent Application Publication
(10) Pub. No.: US 2023/0015539 A1
(43) Pub. Date: Jan. 19, 2023

(54) METHOD OF TREATMENT WITH KCNQ CHANNEL OPENERS

(71) Applicant: Icahn School of Medicine at Mount Sinai, New York, NY (US)

(72) Inventors: James Murrough, New York, NY (US); Eric J. Nestler, New York, NY (US); Ming Hu Han, New York, NY (US)

(21) Appl. No.: 17/781,929

(22) PCT Filed: Dec. 7, 2020
(86) PCT No.: PCT/US2020/063556

§ 371 Date: Jun. 2, 2022

Related U.S. Application Data
(60) Provisional application No. 62/987,781, filed on Mar. 10, 2020.
(60) Provisional application No. 62/945,004, filed on Dec. 6, 2019.

(51) Int. Cl. A61K 31/27; A61P 25/24; A61P 25/22; A61K 9/00

(52) U.S. Cl. CPC A61K 31/27; A61P 25/24; A61P 25/22; A61K 9/0053

(57) ABSTRACT

The present invention relates to methods and compositions for the treatment of psychiatric disorders such as treatment-resistant depression (TRD) with KCNQ (also known as Kv7) channel openers. Also disclosed is a method of inducing or enhancing resilience in a patient by administration of a KCNQ channel opener, such as ezogabine.

This invention was made with government support under Grant No. NIMH R61 MH111932 awarded by NIH/NIMH. The government has certain rights in the invention.

### TECHNICAL FIELD

The present invention relates to methods and compositions for the treatment of psychiatric disorders such as treatment-resistant depression (TRD). More particularly, the invention relates to the administration of KCNQ (also known as Kv7) channel openers such as ezogabine (N-[2-amino-4-(4-fluorobenzylamino)-phenyl] carbamic acid ethyl ester) to treat human patients suffering from TRD, major depressive disorder (MDD), anxiety, anhedonia or post-traumatic stress disorder (PTSD). Also disclosed is a method of increasing resilience in a patient by administration of a KCNQ channel opener such as ezogabine.

### BACKGROUND

Depressive disorders, chief among them major depressive disorder (MDD) and persistent depressive disorder (previously ‘dysthymia’) are among the most disabling medical conditions worldwide. Major depression is a commonly occurring, seriously impairing, and often recurrent mental disorder. Depression ranks first in disability in both high and low income countries among all brain-based disorders, including all psychiatric, neurological and substance disorders. In the United States, the total economic cost of depression in 2012 was estimated to be $188 billion. The World Health Organization ranks MDD as the 4^{th} leading cause of disability worldwide and projects that by 2020 it will be the second leading cause due to currently unexplained increasing prevalence in recent cohorts. Contributing to the problem, a substantial proportion of patients with depression fail to achieve a clinically meaningful improvement in illness severity despite numerous antidepressant trials and augmentation strategies.

Depression is characterized by depressed mood, and markedly diminished interest or pleasure in activities. Other symptoms include feelings of severe despondency and dejection, significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts. A variety of somatic symptoms may also be present. Though depressive feelings are common, especially after experiencing setbacks in life, depressive disorder is diagnosed only when the symptoms reach a threshold and last at least two weeks. Depression can vary in severity from mild to very severe. It is most often episodic but can be recurrent or chronic. Some people have only a single episode, with a full return to premorbid function. However, more than 50 percent of those who initially suffer a single major depressive episode eventually develop another.

Contributing to the problem, a substantial proportion of patients with depression fail to achieve a clinically meaningful improvement in illness severity despite numerous antidepressant trials and augmentation strategies. Especially problematic are patients afflicted with treatment resistant depression. These patients, who have not responded to at least 1 adequate treatment for their depression, are difficult to treat and are often afflicted with comorbidities including for example anhedonia and anxiety. While all forms of depression can be debilitating, those who don't respond to adequate treatments have been found to carry a higher suicide risk than those who do respond to depression treatment. Adequate treatments include antidepressant treatment taken by mouth (such as SSRIs, SNRIs or MAOIs), as well as psychological counseling.

Anhedonia involves various deficits in hedonic function including reduced motivation or inability to experience pleasure. The Diagnostic Manual of the American Psychiatric Association (DSM-5) identifies anhedonia as a symptom of depression and not a separate specific disorder. Anhedonia is a diverse array of deficits in hedonic function, including reduced motivation or ability to experience pleasure. While earlier definitions of anhedonia emphasized the inability to experience pleasure, anhedonia is used by researchers to refer to reduced motivation, reduced anticipatory pleasure (wanting), reduced consummatory pleasure (liking), and deficits in reinforcement learning. In the DSM-5, anhedonia is a component of depressive disorders, substance related disorders, psychotic disorders, and personality disorders, where it is defined by either a reduced ability to experience pleasure, or a diminished interest in engaging in pleasurable activities

The American Psychological Association defines anxiety as an emotion characterized by feelings of tension, worried thoughts and physical changes like increased blood pressure. Patients with anxiety disorders usually have recurring intrusive thoughts or concerns and often have physical symptoms such as sweating or rapid heartbeat.

Post-traumatic stress disorder (PTSD) is a highly debilitating psychiatric disorder characterized by flashbacks, emotional numbness, and insomnia and is associated with functional impairments, physical health concerns and metal health co-morbidities such as depression with a six fold higher risk of suicide. PTSD can result from a catastrophic and threatening event including for example a natural disaster, wartime situation, accident, domestic abuse or violent crime. The disorder is especially prevalent among military combat veterans. Symptoms typically develop within three months, but can emerge years after the initial trauma. Treatment of PTSD is difficult and may include many years of individual and group therapy, as well as administration of antidepressants, anxiolytic drugs, β-adrenergic antagonists, opiates, ketamine (an antagonist of NMDA-type glutamate receptors) or cortisol.

Resilience is the ability to recover quickly from illness, change or misfortune as well as the capacity of a person to cope with stress and catastrophe. A resilient individual will tend to resist the development of stress-related psychological disorders such as depression and PTSD. Resilient people tend to maintain a more positive outlook and cope with stress more effectively, and prevent the development of psychological or psychiatric disorders (i.e., depression, anxiety and PTSD). Resilience may also be defined as the ability to mentally or emotionally cope with a crisis or setback or to return to pre-crisis status quickly. Often defined as the ability to “bounce back” a resilient person is able to withstand and adapt to hardships, including trauma.

Currently available psychotropic medications for the treatment of the abovementioned disorders largely share the same basic pharmacology and mechanism of action based on serendipitous discoveries made decades ago.

What is needed in the art are new medications for treating patients suffering from depressive disorders such as MDD, TRD, PTSD, anxiety and/or anhedonia using mechanistically novel treatments. Also needed are medications for inducing resilience in patients.

### SUMMARY OF THE INVENTION

The present inventors have now unexpectedly discovered that ezogabine (a.k.a. Retigabine), 2-amino-4-(4-fluorobenzylamino)-1-ethoxycarbonylaminobenzene, a KCNQ type K⁺ channel opener (a.k.a. ‘positive modulator’) is effective in treating human patients suffering from TRD, MDD, anxiety, anhedonia and PTSD. KCNQ channels, also known as voltage-gated Kv7 channels, control cell membrane excitability by regulating the flow of potassium (K⁺) across the cell membrane. The KCNQ family of channels comprised of KCNQ1-5, or Kv7.1-7.5. These channels are distributed throughout the central nervous system (CNS) and are implicated in a variety of CNS disorders, including seizure disorder or epilepsy.

It is known that K⁺ channels in the ventral tegmental area (VTA) are an active mediator of resilience in rodent stress models that are relevant to the human conditions of depression, anxiety and PTSD. The KCNQ channels are responsible for the muscarinic currents (M-currents) in neurons and are responsible for repolarizing IC⁺ currents in cardiac tissues (Miceli et al., 2008). The KCNQ channel opener ezogabine has been used in the past as an anticonvulsant in the treatment of epilepsy. Ezogabine is partially selective for the KCNQ2 and 3 channels, which typically dimerize such that ezogabine and its analogs are sometimes referred to simply as ‘KCNQ2/3 channel openers’ or just ‘KCNQ channel openers.’ Known KCNQ channel openers include ezogabine, flupirtine (a selective neuronal potassium channel opener), Acrylamide S-1/S-2, ML213, BMS-204352, N1129, ICA-27243, ICA-069673; Zinc pyrithione, AaTXKβ (2-64), Phenylboronic acid, NS15370, Meclofenac and Diclofenac.

It has now been discovered that KCNQ type K⁺ channel openers, including ezogabine show antidepressant efficacy and in particular are effective in treating TRD. It is believed that KCNQ channel openers function through the potentiation of active resilience mechanisms. Thus, KCNQ channel openers can also function to improve or enhance resilience.

In one aspect, the present invention provides a method for treating a human patient suffering from TRD comprising administering to a patient in need of such treatment an effective amount of KCNQ-selective potassium channel opener to treat said patient and a pharmaceutically acceptable carrier or diluent.

In a further aspect, the patient being treated for TRD with ezogabine has not responded to at least one adequate antidepressant treatments.

In a still further aspect, the present invention provides a method for treating a human patient afflicted with anhedonia and reduced motivation comprising administering to a patient in need of such treatment an effective amount of a KCNQ-selective potassium channel opener to treat said patient and a pharmaceutically acceptable carrier or diluent.

In another aspect, the KCNQ-selective potassium channel opener for treating anhedonia is ezogabine.

In another aspect, 900 mg/day of ezogabine is administered to treat anhedonia.

In yet a further aspect of the invention, 900 mg/day of ezogabine administered in three doses of 300 mg each is administered to treat anhedonia.

In a still further embodiment of the invention, three 300 mg doses of ezogabine are administered to treat a patient suffering from anhedonia with one 300 mg dose being administered in the morning, one 300 mg dose administered mid-day and a final 300 mg dose being administered in the evening.

In another aspect, the KCNQ-selective potassium channel opener for treating anhedonia is a biologically active analog or derivative of ezogabine.

In a still further aspect, the present invention provides a method for treating a patient suffering from anxiety comprising administering to a patient in need of such treatment an effective amount of a KCNQ-selective potassium channel opener to treat said patient and a pharmaceutically acceptable carrier or diluent

In another aspect, the KCNQ-selective potassium channel opener used to treat anxiety is ezogabine.

In another aspect, 900 mg/day of ezogabine is administered to treat anxiety.

In yet a further aspect of the invention, 900 mg/day of ezogabine administered in three doses of 300 mg each is administered to treat anxiety.

In a still further embodiment of the invention three 300 mg doses of ezogabine are administered to treat a patient afflicted with anxiety with one 300 mg dose being administered in the morning, one 300 mg dose administered mid-day and a final 300 mg dose being administered in the evening.

In another aspect, the KCNQ-selective potassium channel opener for treating anxiety is a biologically active analog or derivative of ezogabine.

In yet another further aspect, the present invention provides a method for treating a patient suffering from MDD comprising administering to a patient in need of such treatment an effective amount of a KCNQ-selective potassium channel opener to treat said patient and a pharmaceutically acceptable carrier or diluent

In another aspect, the KCNQ-selective potassium channel opener used to treat MDD is ezogabine.

In another aspect, 900 mg/day of ezogabine is administered to treat MDD.

In yet a further aspect of the invention, 900 mg/day of ezogabine administered in three doses of 300 mg each is administered to treat TRD.

In a still further embodiment of the invention, three 300 mg doses of ezogabine are administered to treat a patient afflicted with MDD with one 300 mg dose being administered in the morning, one 300 mg dose administered mid-day and a final 300 mg dose being administered in the evening.

In yet another aspect, the KCNQ-selective potassium channel opener for treating MDD is a biologically active analog or derivative of ezogabine.

In yet a still further aspect, the present invention provides a method for inducing or enhancing resilience in a patient comprising administering to a patient in need of such treatment an effective amount of a KCNQ-selective potassium channel opener to induce or enhance resilience in said patient and a pharmaceutically acceptable carrier or diluent.

In a further aspect of the invention, the KCNQ-selective potassium channel opener used to enhance or induce resilience is ezogabine.

In another aspect, 900 mg/day of ezogabine is administered to induce or enhance resilience in a patient.

In yet a further aspect of the invention, 900 mg/day of ezogabine administered in three doses of 300 mg each is administered to enhance or induce resilience.

In a still further embodiment of the invention, three 300 mg doses of ezogabine are administered to enhance or induce resilience with one 300 mg dose being administered in the morning, one 300 mg dose administered mid-day and a final 300 mg dose being administered in the evening.

In another aspect, the KCNQ-selective potassium channel opener to enhance or induce resilience is a biologically active analog or derivative of ezogabine.

In a further aspect, the present invention provides a method for treating patients afflicted with PTSD) comprising administering to a patient in need of such treatment an effective amount of a KCNQ-selective potassium channel opener to treat the patient's PTSD.

In another aspect, the KCNQ-selective potassium channel opener administered to treat PTSD is ezogabine.

In another aspect, 900 mg/day of ezogabine is administered to treat PTSD.

In yet a further aspect of the invention, 900 mg/day of ezogabine administered in three doses of 300 mg each is administered to treat PTSD.

In a still further embodiment of the invention three 300 mg doses of ezogabine are administered to treat a patient afflicted with PTSD with one 300 mg dose being administered in the morning, one 300 mg dose administered mid-day and a final 300 mg dose being administered in the evening.

In another aspect, the KCNQ-selective potassium channel opener for treating PTSD is a biologically active analog or derivative of ezogabine.

In yet a still further aspect, the present invention provides a method for treating a patient suffering from persistent depressive disorder (PDD) comprising administering to a patient in need of such treatment an effective amount of a KCNQ-selective potassium channel opener to treat said patient and a pharmaceutically acceptable carrier or diluent.

In a further aspect of the invention, the KCNQ-selective potassium channel opener used to treat PDD is ezogabine.

In another aspect, 900 mg/day of ezogabine is administered to treat PDD in a patient.

In yet a further aspect of the invention, 900 mg/day of ezogabine administered in three doses of 300 mg each is administered to treat PDD.

In a still further embodiment of the invention three 300 mg doses of ezogabine are administered to treat a patient afflicted with PDD with one 300 mg dose being administered in the morning, one 300 mg dose administered mid-day and a final 300 mg dose being administered in the evening.

In another aspect, the KCNQ-selective potassium channel opener for treating PDD is a biologically active analog or derivative of ezogabine.

These and other aspects of the invention will be apparent to those of ordinary skill in the art from the following detailed description. It should be understood, however, that the detailed description and the specific examples, while indicating preferred embodiments of the invention, are given by way of illustration only, because various changes and modifications within the spirit and scope of the invention will be apparent to those skilled in the art from this detailed description.

The details of one or more embodiments of the invention are set forth in the accompanying drawings and the description below. Other features, objects, and advantages of the invention will be apparent from the description and drawings and from the claims.

### BRIEF DESCRIPTION OF DRAWINGS

FIG. 1 is a graph showing the effect of treatment with ezogabine on activation to reward within Ventral Striatum (VS). Change in activation to reward anticipation (gain cue>neutral cue) from baseline to the primary outcome visit (outcome>baseline) in the ezogabine and placebo groups (n=40) within a VS region of interest. The figure shows that ezogabine compared to placebo increased response to reward in the brain as hypothesized. Values reflect means with associated standard error of the mean (SEM). VS, Ventral striatum; NAc, Nucleus Accumbens; ROI, Region of Interest.

FIG. 2 is a graph showing the effect of treatment with ezogabine on anhedonia as measured by the Snaith-Hamilton Pleasure Scale (SHAPS). The figure shows the benefit of ezogabine compared to placebo on improvement in anhedonia as measured by the SHAPS (n=45). Values reflect means with associated standard error of the mean (SEM). SHAPS, Snaith-Hamilton Pleasure Scale. Estimate=−1.55, SEM=0.37; DF=212, t=−4.1, p<0.0001 Cohen's d=0.6.

FIG. 3 is a graph showing the effect of treatment with ezogabine on anticipatory anhedonia as measured by the anticipatory subscale of the Temporal Experience of Pleasure Scale (TEPS). The figure shows the benefit of ezogabine compared to placebo on improvement in anhedonia as measured by the anticipatory subscale of the TEPS (n=45). Values reflect means with associated standard error of the mean (SEM). TEPS, Temporal Experience of Pleasure Scale. Estimate=1.33, SEM=0.39; DF=213, t=3.4, p=0.0008

FIG. 4 is a graph showing the effect of treatment with ezogabine compared to placebo on depression severity as measured by the Montgomery-Asberg Depression Rating Scale (MADRS). The figure shows the change in MADRS scores during a clinical trial of ezogabine compared to placebo in patients with depression (n=45). Values reflect means with associated standard error of the mean (SEM). MADRS, Montgomery—Åsberg Depression Rating Scale. Estimate=1.49 SEM=0.37; DF=213, t=4.04, p<0.0001 Cohen's d=0.7

FIG. 5 is a graph showing the effect of treatment with ezogabine on the Clinical Global Impression-Improvement (CGI-I) scale. The figure shows the benefit of ezogabine compared to placebo of a validated clinician-administered measure of clinical illness improvement; CGI-I, Clinical Global Impression-Improvement. Estimate=1.23 SEM=0.20; DF=4,21, t=6.13, p=<0.0001

FIG. 6 is a graph showing the effect of treatment with ezogabine on the Clinical Global Impression-Severity (CGI-S) scale. The figure shows the benefit of ezogabine compared to placebo of a validated clinician-administered measure of clinical illness severity; CGI-S, Clinical Global Impression-Severity. Estimate=1.1017, SEM=0.045; DF=6.20, t=2.24, p=0.026

FIG. 7 is a graph showing the MADRS change over time: effect of treatment resistance. The figure shows that ezogabine improved depression as measured by the MADRS compared to placebo among patients who suffered from TRD and those without TRD (‘non-TRD’). The line, labeled 1 shows the change over time in TRD patients treated with ezogabine and the line labeled 3 shows change over time in TRD patients treated with placebo. There was a significant effect of treatment specifically among patients with TRD (p=0.024). This suggests that ezogabine can be effective in patients with TRD who are otherwise non-responsive to conventional (i.e. monoamine mechanism) antidepressants. Legend=Y axis: mean MADRS score (lower score is less depressed), X axis: study visit, 1=VO (Baseline), 2—V1, 3—V2, 4—V3, 5—V4, 6—V5 (primary outcome), 7=Legend=Y axis: mean MADRS score (lower score is less depressed), X axis: study visit, 1=VO (Baseline), 2—V1, 3—V2, 4—V3, 5—V4, 6—V5 (primary outcome), 7=V8 (exit visit, following 3 weeks taper).

FIG. 8 is a graph showing the MADRS change over time: effect of treatment resistance within the ezogabine group alone. The data shown is the same as the prior figure, only that only the TRD subgroup treated with ezogabine or placebo are displayed for clarity (the change with treatment in the non-TRD group are not shown). Legend=Y axis: mean MADRS score (lower score is less depressed), X axis: study visit, 1=VO (Baseline), 2—V1, 3—V2, 4—V3, 5—V4, 6—V5 (primary outcome), 7=V8 (exit visit, following 3 weeks taper).

FIG. 9 is a graph showing the Difference in MADRS change: effect of treatment resistance. The figure displays the comparison between change from baseline to end of study in three groups: TRD patients treated with ezogabine, non-TRD patients treated with ezogabine, and placebo. The data show that there is a relatively large effect of ezogabine treatment specifically in patients with TRD; in fact there is an even larger benefit of treatment compared to placebo in the TRD patients than in the non-TRD patients (p=0.015). Legend=Y axis; mean change in MADRS score between primary outcome (V5) and baseline (V0), X axis: 3 groups (TRD randomized to ezogabine, non TRD randomized to ezogabine and placebo), p values reported from independent t-test.

FIG. 10 is a table showing the Difference in MADRS change: effect of treatment resistance, Results from the ANOVA. This exploratory analyses shows that ezogabine has beneficial effects compared to placebo in patients with TRD (p=0.024); nominally the beneficial effect of treatment is larger in patients with TRD compared to those with non-TRD depression. Results from the ANOVA=Highlighted difference between TRD subjects randomized in ezogabine and subjects randomized to placebo.

### DETAILED DESCRIPTION OF THE INVENTION

### Definitions

The term “about” or “approximately” usually means within an acceptable error range for the type of value and method of measurement. For example, it can mean within 20%, more preferably within 10%, and most preferably still within 5% of a given value or range. Alternatively, especially in biological systems, the term “about” means within about a log (i.e., an order of magnitude) preferably within a factor of two of a given value.

Resilience is the ability to mentally or emotionally cope with a crisis or to return to pre-crisis status quickly. Resilience exists in people who develop psychological and behavioral capabilities that allow them to remain calm during crises/chaos and to move on from the incident without long-term negative consequence.

Generally, “treating” or “treatment” of a state, disorder or condition includes: (1) preventing or delaying the appearance of clinical or sub-clinical symptoms of the state, disorder or condition developing in a patient that may be afflicted with or predisposed to the state, disorder or condition but does not yet experience or display clinical or subclinical symptoms of the state, disorder or condition; or 2) inhibiting the state, disorder or condition, i.e., arresting, reducing or delaying the development of the disorder or a relapse thereof (in case of maintenance treatment) or at least one clinical or sub-clinical symptom thereof; or (3) relieving the disorder, i.e., causing regression of the state, disorder or condition or at least one of its clinical or sub-clinical symptoms. The benefit to a subject to be treated is either statistically significant or at least perceptible to the patient or to the physician.

The phrase “pharmaceutically acceptable” refers to molecular entities and compositions that are generally believed to be physiologically tolerable and do not typically produce an allergic or similar untoward reaction, such as gastric upset, dizziness and the like, when administered to a human. The term “pharmaceutically acceptable derivative, e.g. ester, of a compound which upon administration to the recipient is capable of providing (directly or indirectly) a compound or an active metabolite or residue thereof. Such derivatives are recognizable to those skilled in the art, without undue experimentation. Derivatives are described, for example, in Burger's Medicinal Chemistry and Drug Discovery, 5th Edition, Vol 1: Principles and Practice, which is incorporated herein by reference to the extent of teaching such derivatives. Preferred pharmaceutically acceptable derivatives include salts, solvates, esters, carbamates, and phosphate esters. Particularly preferred pharmaceutically acceptable derivatives are salts, solvates, and esters. Most preferred pharmaceutically acceptable derivatives are salts and esters.

The term “carrier” refers to a diluent, adjuvant, excipient, or vehicle with which a composition or pharmaceutical composition, e.g., comprising an effective amount of ezogabine, can be administered. Pharmaceutical and pharmaceutically acceptable carriers include sterile liquids, such as water and oils, including those of petroleum, animal, vegetable or synthetic origin, such as peanut oil, soybean oil, mineral oil, sesame oil, and the like. Water or aqueous saline solutions and aqueous dextrose and glycerol solutions may be employed as carriers, particularly for injectable solutions. Carriers may also include solid dosage forms, including, but not limited to, one or more of a binder (for compressed pills), a glidant, an encapsulating agent, a flavorant, and a colorant. Suitable pharmaceutical carriers are described in “Remington's Pharmaceutical Sciences” by E. W. Martin.

When formulated in a pharmaceutical composition, a therapeutic compound of the present invention can be admixed with a pharmaceutically acceptable carrier or excipient. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human.

Pursuant to the present invention, as used herein, patients suffering from a treatment resistant depressive disorder (TRD) are defined as patients who have not responded to at least one adequate antidepressant treatment in their current major depressive episode. In some embodiments, TRD is defined as patients who have not responded to 2 or more adequate treatments for their depression. For example, according to the The Ruhe Staging Model (J Affect Disord. 2012 March; 137(1-3):35-45. doi: 10.1016/j.jad.2011.02.020. Epub 2011 Mar. 23), a patient who has not responded to one adequate trial of one major class of antidepressants would be considered TRD Stage 1.

By “effective amount” is meant the amount required to ameliorate the symptoms of a disease relative to an untreated patient. The effective amount of active compound(s) used to practice the present invention for therapeutic treatment of a disease varies depending upon the manner of administration, the disorder and its severity, responsiveness of the patient to be treated and the age, body weight, and general health of the subject. Ultimately, the attending physician will decide the appropriate amount and dosage regimen. Such amount is referred to as an “effective” amount. In embodiments of the present invention, an effective amount of KCNQ channel opener such as ezogabine is in the range of from about 0.01 mg/kg to about 1.5 mg/kg. In some aspects, the effective amount is in the range of from about 0.01 mg/kg to about 0.75 mg/kg. In some aspects, the effective amount is in the range of from about 0.75 mg/kg to about 1.5 mg/kg. In some aspects, the effective amount of is in the range of from about 0.5 mg/kg to about 1.2 mg/kg. In some aspects, the effective amount is in the range of from about 0.05 mg/kg to about 0.5 mg/kg. In some aspects, the effective amount is between about 0.2 mg/kg and about 0.4 mg/kg. In one preferred embodiment, an effective amount of ezogabine is 900 mg/day administered in 300 mg doses given three times per day, i.e.one dose in the morning, one dose at midday and one dose in the evening.

KCNQ channel opener means a compound that facilitates ion transmission through potassium channels. This is also referred to a positive modulation of the ion channel or receptor, or positive allosteric modulation. Known KCNQ channel openers include ezogabine, flupirtine (a selective neuronal potassium channel opener), Acrylamide S-1/S-2, ML213, BMS-204352, NH29, ICA-27243, ICA-069673, zinc pyrithione, AaTXKβ (2-64), phenylboronic acid, NS15370, Meclofenac and Diclofenac

Any patient suffering from MDD or TRD may be treated by the methods described herein. Such depressive disorders include but are not limited to any of: treatment resistant depression, anhedonia, anxiety, major depressive disorder, single episode, recurrent major depressive disorder-unipolar depression, seasonal affective disorder, winter depression, bipolar mood disorder, bipolar depression, mood disorder due to a general medical condition with major depressive-like episodes, or mood disorder due to a general medical condition with depressive features, wherein those disorders are resistant to treatment in a given patient. Thus, any patient that presents with one of those disorders can be treated with ezogabine using the methods described herein.

As used herein a chemical analog or simply analog of ezogabine is a compound having a structure similar to that of ezogabine, but differing from it in respect of a certain component. The difference between derivative and analog is that a derivative is a chemical derived from another while an analog is a structural derivative of a parent compound that often differs from it by a single element. Flupirtine is an example of an ezogabine analog (as disclosed in Xiong, et al. Trends in Pharmacological Sciences Vol. 29 No. 2, pp 99-106, 2008.)

The present inventors have now unexpectedly discovered that KCNQ-selective potassium channel openers, including for example ezogabine (Potiga) and their analogs and derivatives are effective agents for treating a wide variety of depressive disorders but are especially effective in treating TRD. This includes patients suffering from Post-Traumatic Stress disorder (PTSD), MDD, anhedonia, anxiety as well as inducing resilience. This was unexpected because before the present invention the only approved therapeutic use for KCNQ channel openers had been as an anticonvulsant agent for use as an adjunctive treatment for partial epilepsies in treatment-experienced adult patients. In addition to its effectiveness in treating a wide variety of depressive disorders, and especially TRD, ezogabine administration led to a rapid onset of relief of the symptoms of the depressive disorders. This is surprising and unexpected because as is well known to those of ordinary skill in the art, antidepressant medications customarily take weeks or months to provide a therapeutic benefit to the patient. Rapid relief is of increased importance to TRD patients as this can reduce the risk of suicide.

Ezogabine selectively binds to and activates KCNQ transmembrane potassium ion channels, enhancing trans-membrane potassium currents mediated by the KCNQ (Kv7.2 to 7.5) family of ion channels. The activation of KCNQ channels is thought to stabilize the resting membrane potential and reduce brain excitability. Derivatives of ezogabine, such as Acrylamide (S)-1, Acrylamide (S)-2, Maxipost, N-phenylanthranilic acid derivatives, Diclofenac, Flufenamic acid, Meclofenamic acid, Flupirtine and zinc pyrithione are also effective in treating TRD as well as MDD, PTSD, anhedonia, anxiety and inducing resilience.

Disturbances within positive valence systems (PVS) drive abnormal reward processing leading to the cardinal symptoms of depression, including anhedonia (the diminished response to pleasure). Depressed patients show characteristic deficits in their capacity to anticipate and respond to pleasurable stimuli, assessed through self-report scales and laboratory-based measures.

Blunted activation during reward anticipation or consumption within brain reward systems that center on the ventral striatum (VS, the VS is also referred to as the Nucleus Accumbens (NAc) in some cases or contexts) have also been consistently linked to depression in general, and in particular, to anhedonia. The present inventors discovered a causal role for the KCNQ-type potassium channel in reversing depressive phenotypes following social defeat stress. Mice manifesting the depressive/anhedonic phenotype (compared to resilient mice) show a failure to increase KCNQ channel activity within the ventral tegmental area (VTA)-VS reward circuit of the brain. This susceptible phenotype can be reversed through (a) overexpression of KCNQ channels, (b) direct VTA injection of KCNQ channel openers, or (c) administering channel openers. Repeated peripheral daily injection of the KCNQ channel opener ezogabine completely reversed the depressive/anhedonic phenotype in susceptible mice. These results identify KCNQ channel openers as a completely novel class of antidepressants.

The present inventors discovered that the KCNQ-selective channel openers including ezogabine demonstrate high selectivity for the target molecular structure (the KCNQ channel) and obtain reliable central nervous system (CNS) exposure. Peripheral administration of ezogabine reversed the anhedonic phenotype via normalization of reward circuit activity in humans suffering from anhedonia and depression. The present inventors examined the clinical and neurocircuit effects of ezogabine in patients with depression and anhedonia. Upon administration to human patients, ezogabine lead to a reduction in anhedonia and related symptoms, and to an increased brain response to reward within the canonical cortico-striatal reward circuit as measured by functional magnetic resonance imaging (fMRI) and a variant of the monetary incentive delay task (MID) (Stern E R, Welsh R C, Fitzgerald K D, Gehring W J, Lister J J, Himle J A, Abelson J L, Taylor S F: Hyperactive error responses and altered connectivity in ventromedial and frontoinsular cortices in obsessive-compulsive disorder. Biol. Psychiatry. 2011; 69:583-591). Ezogabine enhances transmembrane potassium currents mediated by the KCNQ2/3 family of ion channels in order to stabilize neural membranes, likely leading to the observed anticonvulsant effect.

Ezogabine is rapidly absorbed with median time to maximum plasma concentration (Tmax) values between 0.5 and 2 hours and readily crosses the blood brain barrier (BBB) at physiological concentrations. Ezogabine is not subject to P-glycoprotein active transport and free brain concentrations will closely follow free plasma concentrations In clinical trials, free plasma concentrations were approximately 0.4, 0.6, and 0.8 uM for daily doses of 600 mg, 900 mg, and 1200 mg, respectively. Ezogabine is extensively metabolized primarily via glucuronidation and acetylation in humans; the elimination half-life is 7 to 11 hours.

Pursuant to the present invention, ezogabine is administered to patients in need of such treatment in effective amounts broadly ranging between about 1 mg/kg and about 20 mg/kg body weight and preferably between about 5 mg/kg and about 10 mg/kg body weight of the recipient. Ezogabine can be administered parenterally (i. e., intravenously) and preferably, orally for between about 2 and about 8 weeks, dosed daily. In a preferred embodiment, ezogabine is administered orally, three times per day (TID) in 300 mg doses for a total amount of about 900 mg daily. Patients can-be monitored using the clinical measures described in the examples below. The same protocol is also useful in treating anxiety, anhedonia, MDD, TRD, PTSD and also for inducing resilience.

Contemplated for use herein are oral solid dosage forms, which are described generally in Remington's Pharmaceutical Sciences, 18th Ed. 1990 (Mack Publishing Co. Easton Pa. 18042) at Chapter 89). Solid dosage forms include tablets, capsules, pills, troches or lozenges, cachets, pellets, powders, or granules. An exemplary lozenge formulation is described in Chong et al. Clin. Drug Investig. 2009; 29(5): 317-24. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modem Pharmaceutics Edited by G. S. Banker and C. T. Rhodes Chapter 10, 1979. In general, the formulation includes the therapeutic agent and inert ingredients which allow for protection against the stomach environment, and release of the biologically active material in the intestine.

Also contemplated for use herein are liquid dosage forms for oral administration, including pharmaceutically acceptable emulsions, solutions, suspensions, and syrups, which may contain other components including inert diluents; adjuvants, wetting agents, emulsifying and suspending agents; and sweetening, flavoring, coloring, and perfuming agents.

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine, e.g., by the use of an enteric coating. Examples of the more common inert ingredients that are used as enteric coatings are cellulose acetate trimellitate (CAT), hydroxypro-pylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used. The formulation of the material for capsule administration could also be as a powder, lightly compressed plugs, or even as tablets. These therapeutics could be prepared by compression.

One may dilute or increase the volume of the therapeutic agent with an inert material. These diluents could include carbohydrates, especially mannitol, lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic agent into a solid dosage form. Materials used as disintegrants include but are not limited to starch, including the commercial disintegrant based on starch, Explotab, Sodium starch glycolate, Amberlite, sodium carboxymethyl cellulose, ultramylopectin, sodium alginate, gelatin, orange peel, acid carboxymethyl cellulose, natural sponge and bentonite may all be used. The disintegrants may also be insoluble cationic exchange resins. Powdered gums may be used as disintegrants and as binders, and can include powdered gums such as agar, Karaya or tragacanth. Alginic acid and its sodium salt are also useful as disintegrants. Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the peptide (or derivative).

An antifrictional agent may be included in the formulation to prevent sticking during the formulation process. Lubricants may be used as a layer between the peptide (or derivative) and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

In another alternative embodiment, administration comprises transdermal administration. Such treatment may be administered alone or may be supplemented with other antidepressant therapies as described herein. Transdermal administration includes passive or active transdermal or transcutaneous modalities, including, for example, patches and iontophoresis devices, as well as topical application ofpastes, salves, or ointments.

The compositions and formulation described herein may be administered by a health professional or by a patient. Patient self-administration of KCNQ channel openers such as ezogabine is expressly contemplated. Intranasal administration and administration via transdermal patch are suited to patient self-administration.

Additional Active Ingredients

Formulations for use in the methods described herein can include other therapeutically or pharmacologically active ingredients in addition to ezogabine such as but not limited to agents used for antidepressant therapy. Such agents include, but are not limited to, antidepressants: e.g., biogenic amine non-selective reuptake inhibitors, e.g., tricyclic anti-depressants like imipramine; serotonin selective reuptake inhibitors like fluoxetine (Prozac); monoamine oxidase inhibitors (MAO-I) like phenelezine; other types of anti-depressant medications including atypical antidepressants. Antidepressants augmentation with other medications e.g., lithium, T3, T4, etc. Other treatment modalities with antidepressant effects: electro-convulsive treatment (ECT); light therapy, psychotherapy e.g., cognitive or interpersonal therapy for PTSD.

In addition, administration of drugs reported to ameliorate or exacerbate the symptoms of a neuropsychiatric disorder, include but are not limited to antidepressant compounds such as lithium salts, carbamazepine, valproic acid, lysergic acid diethylamide (LSD), p-chlorophenylalanine, p-propyidopacetamide dithiocarbamate derivatives e.g., FLA 63; anti-anxiety drugs, e.g., diazepam; monoamine oxidase (MAO) inhibitors, e.g., iproniazid, clorgyline, phenelzine, tranylcypromine, and isocarboxazid; biogenic amine uptake blockers, e.g., tricyclic antidepressants such as desipramine, imipramine and amitriptyline; atypical antidepressants such as mirtazapine, nefazodone, bupropion; serotonin reuptake inhibitors e.g., fluoxetine, venlafaxine, and duloxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), S and dibenzodiazepines (e.g., clozapine); benzodiazepines; dopaminergic agonists and antagonists e.g., L-DOPA, cocaine, amphetamine, a-methyl-tyrosine, reserpine, tetra-benazine, benztropine, pargyline; noradrenergic agonists and antagonists e.g., clonidine, phenoxybenzamine, phentolamine, tropolone.

In another embodiment of the treatment methods, the compositions administered comprise compounds, in particular drugs, reported to ameliorate or exacerbate the symptoms of oxidative stress disorder. Such compounds include reduced glutathione (GSH), glutathione precursors, e.g., N-acetylcysteine; antioxidants, e.g., vitamins E and C, beta carotene and quinones; inhibitors of lipid membrane peroxidation, e.g., 21-aminosteroid U74006F (tirilazad mesylate), and lazaroids; antioxidants such as mazindol; 2c dizocilpine maleate; selegiline; sulfhydryls N-acetyleysteine and cysteamine; dimethylthiourea; EUK-8 a synthetic, low molecular salenmanganese complex; synthetic manganese-based metalloprotein superoxide dismutase mimic, SC52608; free radical scavengers or suppressors, e.g., pegorgotein, tocotrienol, tocopheral, MDL 74, 18, LY231617, MCI-186, AVS (nicaraven), allopurinol, rifampicin, oxypurinol, hypochlorous acid or recombinant human Cu, Zn-SOD.

Effective amounts of ezogabine in compositions including pharmaceutical formulations, include doses that partially or completely achieve the desired therapeutic, prophylactic, and/or biological effect. In a specific embodiment, an effective amount of ezogabine administered to a subject with TRD is effective for treating one or more signs or symptoms of TRD. The actual amount effective for a particular application depends on the condition being treated and the route of administration. In one preferred embodiment, 900 mg/day of ezogabine delivered in 3 equal doses of 300 mg. each, is effective to treat TRD.

The method disclosed herein provides for administration of a therapeutically effective dose of ezogabine, e.g. a dose effective to treat TRD. Specific dosages may be adjusted depending on conditions of disease, i.e., the severity of the subject's condition, the age, body weight, general health conditions, sex, and diet of the subject, dose intervals, administration routes, excretion rate, and combinations of drugs. Any of the dosage forms described herein containing effective amounts of ezogabine, either alone or in combination with one or more active agents, are well within the bounds of routine experimentation and therefore, well within the scope of the instant invention.

An initial dose may be larger, followed by smaller maintenance doses. The dose may be administered as infrequently as weekly or biweekly, or fractionated into smaller doses and administered daily, several times daily, semi-weekly, bi-weekly, quarterly, etc., to maintain an effective dosage level. Preliminary doses can be determined according to animal tests, and the scaling of dosages for human administration can be performed according to art-accepted practices. In certain embodiments, a subject may be administered 1 dose, 2 doses, 3 doses, 4 doses, 5 doses, 6 doses or more of an ezogabine containing composition described herein. However, other ranges are possible, depending on the subject's response to the treatment. Moreover, an initial dose may be the same as, or lower or higher than subsequently administered doses of ezogabine.

The number and frequency of doses may be determined based on the subject's response to administration of the composition, e.g., if one or more of the patient's symptoms improve and/or if the subject tolerates administration of the composition without adverse reaction; in some subjects, a single dose is sufficient, other subjects may receive a daily, several times a day, every other day, several times per week, weekly, biweekly, semi-weekly, or monthly administration of a composition containing ezogabine as described herein. The duration and frequency of treatment will depend upon the subject's response to treatment, i.e., if the subject's condition and/or one more symptoms of e.g. TRD, MDD or PTSD improves.

Preferably, the effective dose of ezogabine is titrated under the supervision of a physician or medical care provider, so that the optimum dose for the particular application is accurately determined Thus, the present disclosure provides a dose suited to each individual subject (e.g., patient).

Once a dosage range is established, an advantage of compositions for intranasal administration of ezogabine and methods of treatment via intranasal administration is that the patient can administer (e.g., self-administer) ezogabine on an as-needed, dose-to-effect basis. Thus, the frequency of administration is under control of the subject.

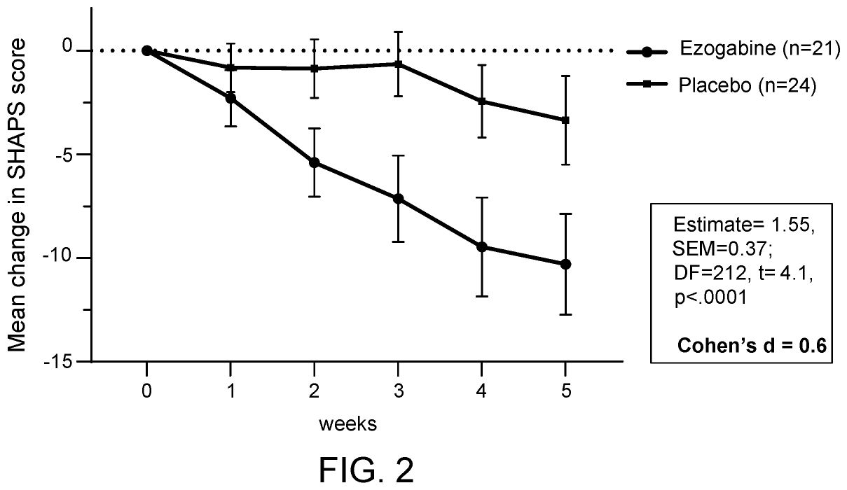

As shown in FIG. 2, oral administration of 300 mg of ezogabine, three times per day (TID) for a total amount of about 900 mg daily for 5 weeks successfully treated patients suffering from anhedonia as determined by the SHAPS scale. As set forth in Example 1 below, subjects treated with ezogabine showed a significant improvement of anhedonia symptoms, as measured by clinical measures of depression.

In one preferred embodiment, an oral ezogabine formulation is used for an outpatient group of depressed patients who are considered treatment resistant, i.e., having had at least 1 adequate treatment failure, and in many cases will have failed at least 2 or more prior antidepressant treatments. The oral formulation will eliminate the necessity of patient presentation to a hospital or clinic for intravenous administration. The patient can take oral ezogabine in their own home, with no need for a needle stick. Thus, the acceptability of the treatment for patients will improve compliance. The patient may be one that is at least a moderately treatment resistant patient, who is seeking new options for the rapid and safe reduction of depressive symptoms. The physician would monitor the patient as an outpatient, and could adjust dosage as necessary. Similar treatment regimes are employed to treat MDD, TRD, anhedonia, anxiety, PTSD or to induce resilience.

The present invention is described below in working examples which are intended to further describe the invention without limiting the scope thereof.

In the examples below, the following clinical measures of depression were utilized.

The well-known Montgomery-Asberg Depression Rating Scale (MADRS) is employed to capture change in overall depressive severity. The MADRS has demonstrated good reliability, validity, and is sensitive to change in patients with depression. The primary clinical outcome is a MADRS subscale composed of items 7 and 8 from the MADRS that capture symptoms of reduced motivation, low energy, and loss of interest or pleasure, which are termed “MADRS”.

The Temporal Experience of Pleasure Scale (TEPS) is designed to capture the separate reward processing components of anticipation and consumption, mapping the PVS constructs of AM/reward expectancy and IRRA/SSRA, respectively.

The TEPS is an 18-item self-report measurement of anticipatory (10 items) and consummatory (eight items) components of anhedonia which consists of a series of statements that must be rated according to how accurate they are for the individual. The scale differentiates the role of anticipatory pleasure (‘wanting’) from consummatory pleasure (‘liking’). The TEPS-Anticipatory sub-scale is a validated measure of anticipation of reward (higher scores are better); the TEPS-Consummatory sub-scale is a validated measure of response to receipt of reward.

Two additional instruments include the Specific Loss of Interest and Pleasure Scale (SLIPS) and the Anticipatory and Consummatory Interpersonal Pleasure Scale (ACIPS) which offer additional unique features, including specificity for anhedonia in the context of depression, and a focus on social aspects of reward.

The Quick Inventory of Depressive Symptomatology-Self-Report (QIDS-SR) is a validated self-report of depression.

The Probabilistic Reward Task (PRT, Pizzagalli D A, Jahn A L, O'Shea J P: Toward an objective characterization of an anhedonic phenotype: a signal-detection approach. Biol. Psychiatry. 2005; 57:319-327) is a signal detection test that provides an objective assessment of reward learning and was completed by all study participants on the baseline (V0) and primary outcome visit (V5). The task consisted of blocks and is completed on a computer wherein participants are presented with ‘rich’ and ‘poor’ stimuli and are instructed to press an appropriate button to decide which stimulus is present. The degree of response bias toward the more frequently reinforced stimulus is used for operationalizing sensitivity to reward. Healthy subjects reliably develop a response bias for the rich stimulus, regardless of which stimulus is actually presented. Subjects with depression, however, tend to respond similarly to both stimuli, and fail to develop this bias for the more frequently reinforced stimulus, thus indicating decreased responsiveness to rewards.

The CGI-S/I will provide an overall clinician-determined summary measure that takes into account all available information, including knowledge of the subject's history, psychosocial circumstances, symptoms, behavior, and the impact of the symptoms on the subject's ability to function. The CGI evaluates the severity of psychopathology from 1 to 7. The CGI-S is a widely administered clinician rated global measure of overall illness severity. Subjects are rated on a 1-7 scale where 1 corresponds to “Normal, Not at All Ill”, 2 is “Borderline Mentally Ill”, the anchor for 3 is “Mildly Ill”, the anchor for 4 is “Moderately Ill”, 5 is “Markedly Ill”, 6 is “Severely Ill”, and 7 is “Among the Most Extremely Ill Patients”. It will be administered at all subject visits. The CGI-I is a widely administered clinician rated global measure of the degree of improvement from the initial assessment in subject overall illness severity. Subjects are rated on a 1-7 scale where 1 corresponds to “Very Much Improved”, 2 is “Much Improved”, the anchor for 3 is “Minimally Improved”, the anchor for 4 is “No Change”, 5 is “Minimally Worse”, 6 is “Much Worse”, and 7 is “Very Much Worse”. It will be administered at all subject visits.

### Example 1: A Multi-Center, Parallel Arm, Double-Blind, Randomized, Placebo-Controlled, Clinical Trial of Ezogabine

The study procedures were conducted at the Icahn School of Medicine at Mount Sinai in New York City and at Baylor College of Medicine in Houston, Tex. Participants were between the ages of 18 and 65 and meet DSM-V criteria for major depressive disorder (MDD) or persistent depressive disorder (PDD, a chronic form of depression), as assessed by a trained rater using the Structured Clinical Interview for Diagnostic and Statistical Manual of Mental Disorders-Fifth Edition Text Revision (DSM-V-TR) Axis I Disorders, Patient Edition (SCID-I/P).

Following screening (Day −28 to Day −2), screening data was reviewed by the site PI to determine subject eligibility. Subjects who met all inclusion criteria and none of the exclusion criteria entered into the study. All eligible participants returned to the clinics to complete the baseline assessments and undergo randomization (Day 0, Baseline). Baseline assessments included clinical evaluations, the PRT and fMRI scanning (details below). Participants who completed these assessments were then randomly assigned to one of the treatment arms (ezogabine or placebo) in a 1:1 fashion under double-blind conditions and entered the treatment period. Randomization was balanced by using randomly permuted blocks and stratified by center. A computer-generated randomization scheme developed by the coordinating site assigned a unique treatment code, which dictated the treatment assignment and matching study drug kit for each subject. The randomization list was provided by the research pharmacist at the coordinating site to the research pharmacist at the collaboration site. The identity of test and control treatments were not known to investigators, research staff, or patients.

During the treatment period, ezogabine was titrated according to the FDA guidelines until reaching the maximum target dose of 300 mg three times daily (900 mg/day) at week four. At each visit, participants completed self-report and clinician-administered rating scales performed by trained raters, and met with a study psychiatrist who assessed suicidal thinking or behavior, adverse events (AE), and changes in concomitant medications. The treatment period consisted of five study visits, which culminated in the primary outcome visit, where participants completed clinician administered and self-reports questionnaires and underwent the second and final fMRI scan and PRT assessment. Following this visit, participants were instructed to taper the study medication over three weeks according to the FDA-recommended guidelines (during which they received weekly phone calls from a member of the study team to assess compliance and side effects) and returned to the clinic for a final study exit visit. The total duration of subject participation was up to 14 weeks.

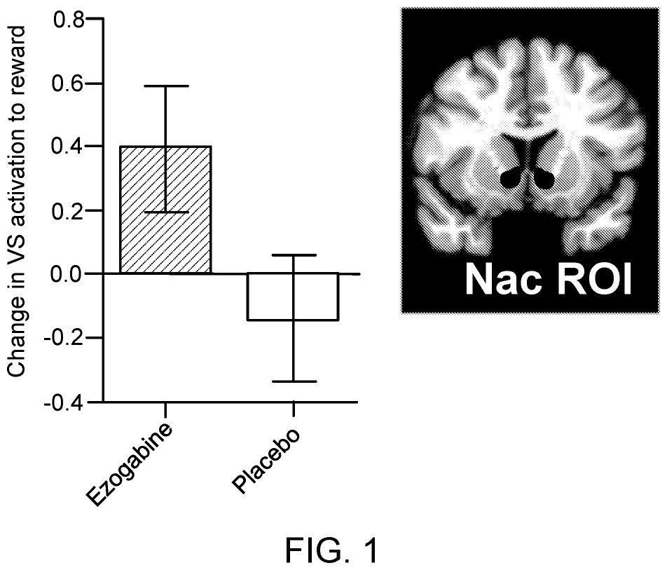

The primary outcome measure of the study was a change in activation within the bilateral ventral striatum (VS, FIG. 1) from baseline (Study Visit 0) to the primary outcome visit (Study Visit 5) as measured by functional magnetic resonance imaging (fMRI) during the Incentive Flanker Task (IFT), a variant of the monetary incentive delay task (MID) as described above. Secondary outcomes measures included change in activation within other regions of the reward cortico-striatal circuit as measured by fMRI during the IFT and change in clinical symptoms of anhedonia (as measured by the SHAPS Temporal Experience of Pleasure Scale [TEPS], Specific Loss of Interest and Pleasure Scale [SLIPS], Anticipatory and Consummatory Interpersonal Pleasure Scale [ACIPS]) and on depressive symptoms assessed through ratings scales (Montgomery-Asberg Depression Rating Scale [MADRS], Quick Inventory of Depressive Symptomatology-Self-Report) from baseline to the primary outcome visit. Additional secondary outcome included change in behavioral symptoms of anhedonia through a computer-based task, the Probabilistic Reward Task (PRT), and global illness improvement and severity evaluated through changes in CGI-I and CGI-S scores. Adverse events were summarized according to the Medical Dictionary for Regulatory Activities (MedDRA) system organ class and preferred terms. Safety and tolerability were assessed by discontinuation rate, frequency of adverse events, and change in score on the well-known Columbia Suicide Severity Rating Scale (Arch Suicide Res. 2018 April-June; 22(2):278-294. doi: 10.1080/13811118.2017.1334610. Epub 2017 Jul. 17. Use of the Columbia-Suicide Severity Rating Scale (C-SSRS) to Classify Suicidal Behaviors. Interian A, Chesin M, Kline A, Miller R, St Hill L, Latorre M, Shcherbakov A, King A, Stanley B).

### Results

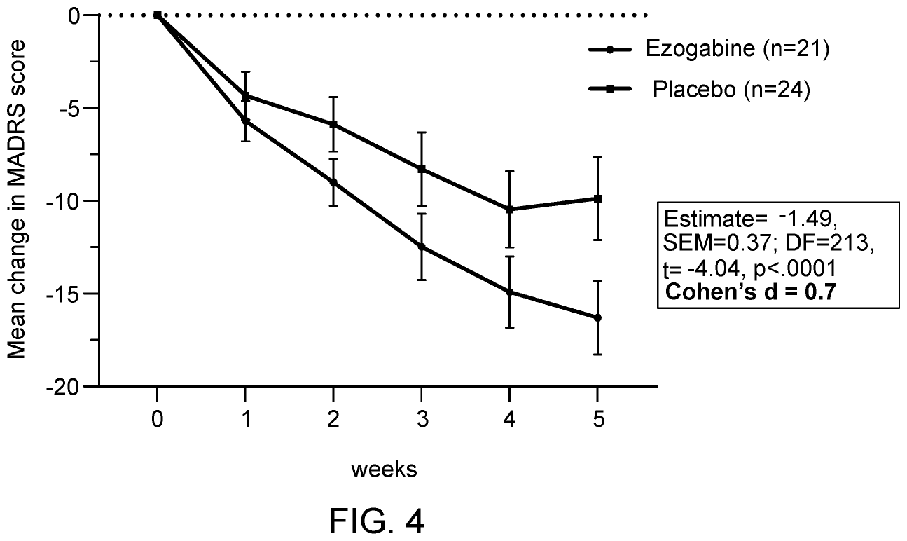

Ezogabine treatment produced significantly greater improvement on symptoms of depression than placebo, as measured by the MADRS and QIDS-SR. At the primary outcome visit, the mean change from baseline in MADRS score was 5.9±8.8 points in the ezogabine group and −8±10.4 points in the placebo group. During the trial, ezogabine produced markedly greater improvement in depressive severity on the MADRS (Estimate=−1.49, SEM=0.37; DF=213, t=−4.04, p<0.0001) (FIG. 4). Likewise, ezogabine produced a significant reduction in QIDS-SR scores compared to placebo (Estimate=−0.65, SEM=0.21; DF=213, t=−3.1, p=0.002).

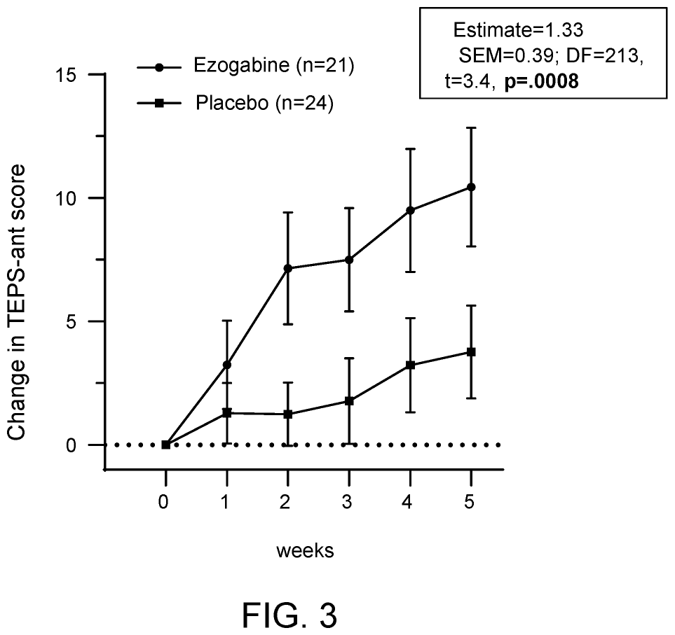

Subjects treated with ezogabine also showed a significant improvement on anhedonia symptoms, as measured through anticipatory subscale of the TEPS (sometimes referred to as ‘TEPS-ant’) (FIG. 3) and SHAPS (FIG. 2). During the trial, ezogabine showed a greater change in SHAPS scores compared to placebo (FIG. 2) (estimate=−1.55, SEM=0.37; DF=212, t=−4.1, p<0.0001) consistent with a medium effect size (Cohen's d=0.6). In particular, the effect of ezogabine on anhedonic symptoms appeared associated primarily with the anticipatory component of anhedonia, as measured by the TEPS-ant (Estimate=1.33, SEM=0.39; DF=213, t=3.4, p=0.0008, FIG. 3) and less with the consummatory component of pleasure (TEPS-con, estimate=0.64, SEM=0.32; DF=213, t=1.9, p=0.05). Ezogabine also showed significant improvement in other measures of anhedonia, such as the ACIPS (Estimate=2.77, SEM=0.72; DF=213, t=3.8, p=0.0002), and the SLIPS (Estimate=−1.78, SEM=0.53; DF=213, t=−3.3, p=0.0009), compared to placebo.

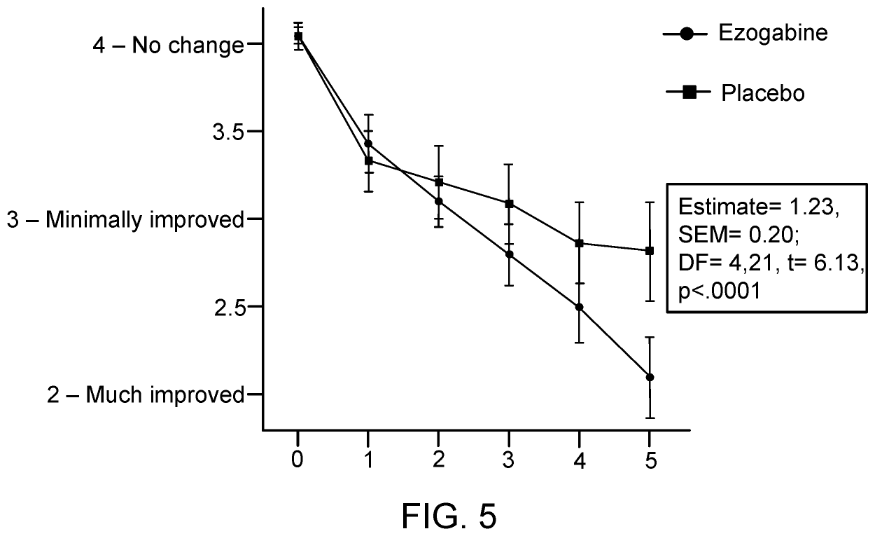

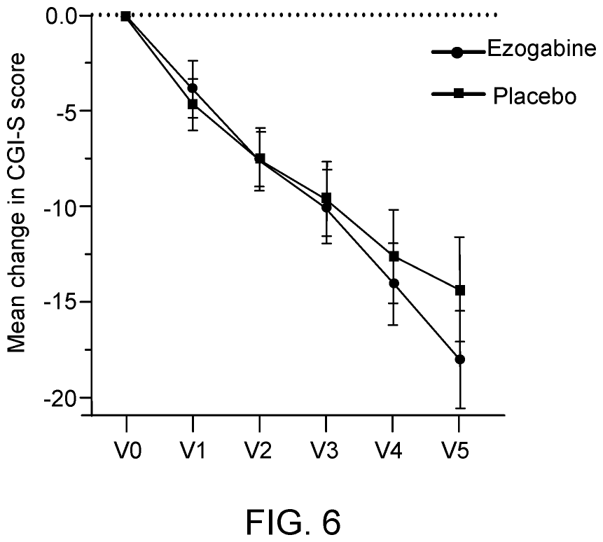

Finally, ezogabine showed lower scores in the clinical global impression scales (CGI), for both improvement (CGI-I, Estimate=−0.14, SEM=0.05; DF=214, t=−2.9, p=0.004, FIG. 5), and severity (Estimate=−0.1, SEM=0.04; DF=214, t=−2.2, p=0.026, FIG. 6), compared to placebo.

### Example 2: Administration of Ezogabine to Patients Suffering from Treatment Resistant Depression

A sub-group of patients with depression were identified who were enrolled in the clinical trial who met one accepted definition of TRD (for example see Treatment-resistant depression: definitions, review of the evidence, and algorithmic approach, McIntyre R S et al. J Affect Disord. (2014)), which is a failure to respond to at least one prior adequate antidepressant medication. Patients designated as being afflicted with TRD in the current study had failed to respond to at least one prior antidepressant medications in the current episode as measured by a validated instrument, the Antidepressant Treatment History Form (ATHF). Among all patients randomized, N=9 in the ezogabine group and N=11 in the placebo group were identified as having TRD.

In this example, treatment resistant depression is defined as a depressed patient that has received at least one adequate prior treatment as captured by the Antidepressant Treatment History Form (ATHF). Patients in Example 1 with treatment resistant depression (TRD) were selected and randomized into 4 groups as follows and treated with either ezogabine or placebo. Treatment resistance is defined as at least one probable treatment failure as captured and by the Antidepressant Treatment form (AHTF). The randomized groups contained: 9 TRD patients randomized to ezogabine, 11 non-TRD patients randomized to ezogabine, 11 TRD patients randomized to placebo and 11 non-TRD patients randomized to placebo.

Ezogabine was administered at a concentration of 900 mg/day in 300 mg doses given three times per day, i.e. in the morning, midday and in the evening for the study duration (five weeks).

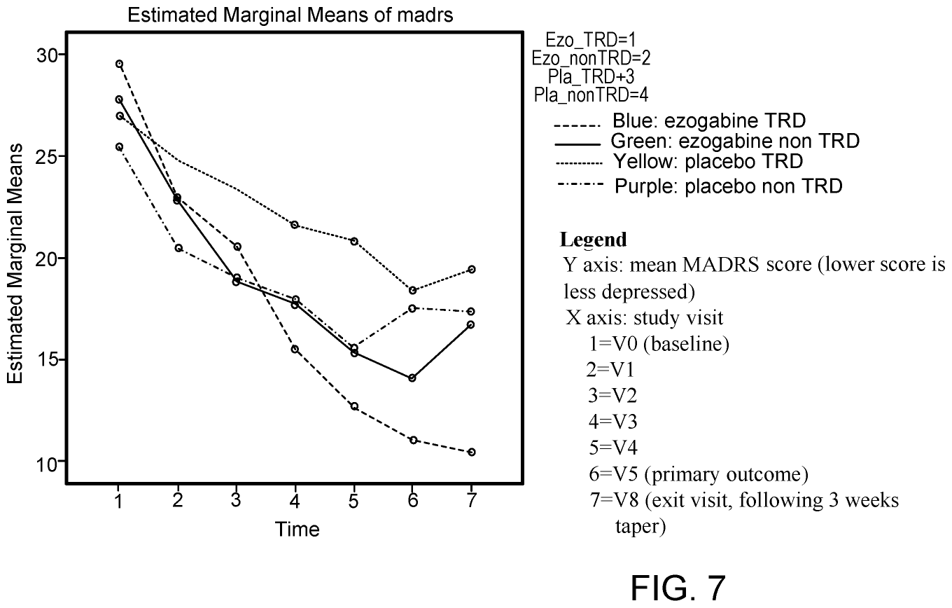

The results are set forth in FIGS. 7-10.

FIG. 7 illustrates the change over time in the Montgomery Asberg Depression Rating Scale score (Estimated Marginal Mean of MADRS) of patients in all four randomized groups. The patients in the ezogabine TRD group had the greatest improvement in their MADRS scores and more significantly began to show significant improvement after a single treatment.

The figure shows that ezogabine improved depression as measured by the MADRS compared to placebo among patients who suffered from TRD and those without TRD (‘non-TRD’). The line labeled 1 shows the change over time in TRD patients treated with ezogabine and the line labeled 3 shows change over time in TRD patients treated with placebo. There was a significant effect of treatment specifically among patients with TRD (p=0.024). This suggests that ezogabine can be effective in patients with TRD who are otherwise non-responsive to conventional (i.e. monoamine mechanism) antidepressant

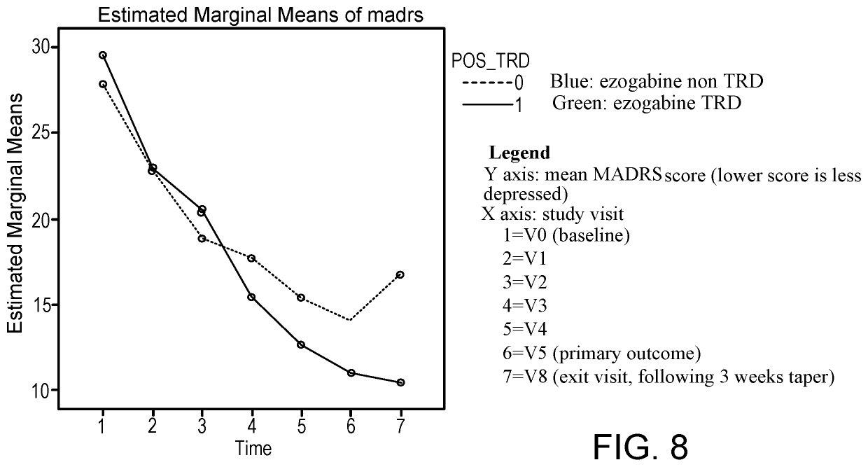

FIG. 8 illustrates the effect on treatment resistance within the ezogabine group alone. The data shown is the same as the prior figure, only that only the TRD subgroup treated with ezogabine or placebo are displayed for clarity (the change with treatment in the non-TRD group are not shown).

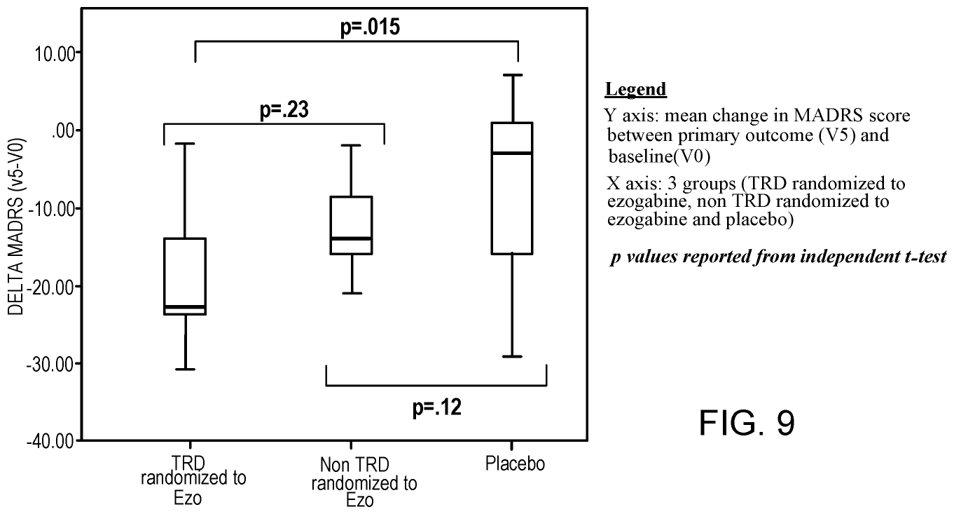

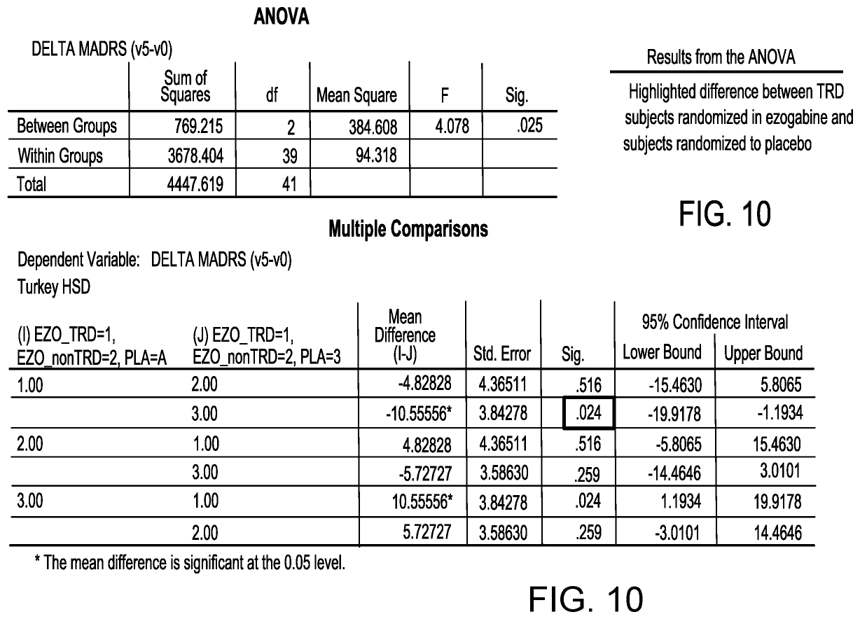

FIG. 9 depicts the change in MADRS scores of TRD and non-TRD patients between primary outcome (V5) and baseline (V0). FIG. 10 illustrates the difference in MADRS score change of TRD and non-TRD patients in the study. The difference between TRD subjects randomized to ezogabine and subjects randomized to placebo is highlighted.

FIG. 10 is a table showing the Difference in MADRS change: effect of treatment resistance, Results from the ANOVA. This exploratory analyses shows that ezogabine has beneficial effects compared to placebo in patients with TRD (p=0.024); nominally the beneficial effect of treatment is larger in patients with TRD compared to those with non-TRD depression.

### Example 3: Use of Ezogabine to Treat an Anxiety Disorder

Patients diagnosed with an anxiety disorder are treated by orally administering 900 mg of ezogabine (300 mg, TID) for the study duration. Patients experience rapid and sustained alleviation of their symptoms of anxiety such as feelings of tension, worried thoughts and physical changes such as increased blood pressure following ezogabine treatment.

### Example 4: Use of Ezogabine to Treat PTSD

Patients diagnosed with PTSD are treated by orally administering 900 mg of ezogabine (300 mg, TID) daily for the study duration. PTSD symptoms such as flashbacks, hypervigilance, nightmares, distancing from others, emotional numbness, insomnia and emotional and physical reactions triggered by reminders of the trauma are alleviated after treatment with ezogabine.

### Example 5: Use of Ezogabine to Induce Resilience

Patients enduring stressful life situations such as job loss, financial problems, illness or divorce are treated by orally administering 900 mg of ezogabine (300 mg, TID) daily for the study duration. After treatment, patients are better at managing their emotional health and wellness.

A number of embodiments of the invention have been described. Nevertheless, it will be understood that various modifications may be made without departing from the spirit and scope of the invention. Accordingly, other embodiments are within the scope of the following claims.

It is further to be understood that all values are approximate, and are provided for description. Patents, patent applications, publications, product descriptions, and protocols are cited throughout this application, the disclosures of which are incorporated herein by reference in their entireties for all purposes.

1. A method for treating a human patient suffering from treatment resistant depression (TRD) comprising
administering to a patient in need of such treatment a pharmaceutical formulation comprising an effective amount of a KCNQ-selective potassium channel opener to treat said patient and a pharmaceutically acceptable carrier or diluent.

2. The method of claim 1, wherein said KCNQ-selective potassium channel opener. is ezogabine.

3. The method of claim 1, wherein said patient has not responded to two adequate antidepressant treatments.

4. The method of claim 1, wherein said effective amount is 900 mg per day.

5. The method of claim 4, wherein said effective amount is three 300 mg of ezogabine administered three times per day.

6. The method of claim 1 wherein said KCNQ-selective potassium channel opener is a biologically active analog or derivative of ezogabine.

7. The method of claim 1 wherein, the patient being treated for TRD with ezogabine has not responded to at least one adequate antidepressant treatment.

8-32. (canceled)

33. A method for treating a patient suffering from persistent depressive disorder (PDD) comprising administering to a patient in need of such treatment a pharmaceutical formulation comprising an effective amount of a KCNQ-selective potassium channel opener, and a pharmaceutically acceptable carrier or diluent.

34. The method of claim 33 wherein said KCNQ-selective potassium channel opener is ezogabine.

35. The method of claim 34, wherein said effective amount is 900 mg per day.

36. The method of claim 35, wherein said effective amount is three 300 mg of ezogabine administered three times per day.

37. The method of claim 33 wherein said KCNQ-selective potassium channel opener is a biologically active analog or derivative of ezogabine.

38-44. (canceled)

45. The method of claim 6, wherein said patient has not responded to at least one adequate antidepressant treatments.

46. The method of claim 6 which comprises orally administering ezogabine to the patient.

47. The method of claim 6 which comprises administering ezogabine parenterally to the patient.

48. The method of claim 1 which comprises administering between about 5 mg/kg and about 20 mg/kg body weight of KCNQ Channel opener to the patient.

49. The method of claim 1 which comprises administering the KCNQ Channel opener with an enteric coating.

50. The method of claim 1 further comprising administering an anti-depressant agent to the patient.

51. The method of claim 50 wherein the antidepressant is a serotonin selective reuptake inhibitor.

52. The method of claim 1 further comprising administering a compound that ameliorates or exacerbates symptoms of oxidative stress disorder.

53. The method of claim 1 which comprises administering an initial dose of said KCNQ channel opener followed by smaller maintenance doses.
